(19) United States
(12) Patent Application Publication
(10) Pub. No.: US 2014/0322371 A1
(43) Pub. Date: Oct. 30, 2014

(54) BIXA ORELLANA COMPOSITION FOR TREATING MACULAR DEGENERATION

(71) Applicants: Stanislas Veillet, Savigny sur Orge (FR); Rene Lafont, Paris (FR); Valerie Fontaine, Paris (FR); Jose-Alain Sahel, Paris (FR)

(72) Inventors: Stanislas Veillet, Savigny sur Orge (FR); Rene Lafont, Paris (FR); Valerie Fontaine, Paris (FR); Jose-Alain Sahel, Paris (FR)

(21) Appl. No.: 14/117,461

(22) PCT Filed: May 14, 2012
(86) PCT No.: PCT/FR2012/000193

§ 371 Date: Mar. 14, 2014

(30) Foreign Application Priority Data
May 13, 2011 (FR) 11 54172

(51) Int. Cl. A61K 36/185; A61K 31/353; A23L 1/30; A61K 31/352

(52) U.S. Cl. CPC A61K 36/185; A61K 31/352; A61K 31/353; A23L 1/3002
USPC 424/776; 514/456

(57) ABSTRACT

A composition includes an extract of seeds of Bixa orellana for the photoprotection of the retinal pigment epithelium in mammals. The use of such a composition for the treatment of macular degeneration related to age (AMD) in mammals is also described.

The invention relates to the use of compounds and a composition for preventing certain diseases of the retina.

The invention aims to improve the vision of diseased people, or at least stabilize the development of these diseases.

Age-related macular degeneration, or AMD, is a chronic degenerative retinal disease, progressive and disabling, affecting the elderly and whose origin is multifactorial (Bellmann and Sahel, 2007). In France, it is the major cause of visual impairment above 50 years and the number of people affected is estimated at one million. A disease with genetic predisposition (Fajnkuchen and Cohen, 2008), it is responsible for a growing number of cases of bad vision, proportional to the increase in life expectancy. This disease affects a small part of the retina, the macula, an area that used to fix objects, to read, recognize faces and discern colors. AMD is most likely polygenic with the intervention of risk factors such as prolonged exposure to light, high blood pressure, hypercholesterolemia and smoking. There are two types of AMD, the dry or atrophic form which represents 80% of cases, and the wet form. Only the latter, which is characterized by the appearance of new blood vessels behind the retina, can presently benefit from treatments.

The pathophysiological mechanisms of AMD are still poorly known, but the involvement of processes of intoxication leading to the death of the retinal pigmentary epithelium (RPE) cells has been established over recent years. Indeed, during aging, these cells may exhibit dysfunctions related to lysosomal accumulation of protein-lipid complexes called lipofuscin granules. These granules are progressively formed by the accumulation of undegraded proteins and lipids originating from the phagocytosis by the RPE of the outer segments of photoreceptors (Finnemann et al., 2002). Lipofuscin also includes cytotoxic derivatives of visual cycle pigments, such as A2E, which is formed by a combination of two molecules of trans-retinal with an ethanolamine molecule. Under the effect of blue light, A2E is oxidized and induces protein, lipid and DNA oxidation, causing a significant oxidative stress in the RPE cells during aging (Kim et al. 2006). Attempts at prevention or treatment of dry AMD are based on nutritional supplementation with substances that reduce the accumulation and/or adverse effects of A2E (Dubernard et al, 2006; Souied et al. 2007; Dutot et al, 2008; Lecerf, 2009; Cohen et al, 2010; Lecerf and Desmettre, 2010).

Given the very probable role of this mechanism in the development of AMD, the inventors used an in vitro cellular model of induced phototoxicity by the association of a treatment with A2E and illumination by blue light on RPE primary cell cultures wherein cell survival was measured. This model was developed by the Institute of Vision and allows for screening molecules aimed to discover new candidates for the treatment of dry AMD. This original model is closer to the “physiological” situation than cell lines commonly used in other laboratories (Dunn et al., 1996), because the cells used already contain protective substances originating from the animal diet and thus are not in a situation of “deficiency”, and their disturbance is induced by the addition of A2E.

The invention thus provides an opportunity to find an alternative treatment to already existing ones.

Specifically, the inventors have discovered that the prior incubation of cells with certain molecules greatly reduces the cell death caused by illumination with blue light of RPE cells pretreated with A2E.

According to the invention, these molecules are present in a urucum extract, or are derivatives of gallic acid or compounds of the family of anthocyanidins.

One aspect of the invention therefore relates to a composition comprising an extract of urucum seeds for photoprotection cells of retinal pigment epithelium in a mammal. The urucum or achiote, or Bixa orellana is a tree or shrub of tropical America. It produces red fruit filled with seeds thorns.

In the context of the invention, the term “seed extract of Bixa orellana” is an extract prepared from the outer coat of the seeds, that is to say, the waxy substance covering the seeds of Bixa orellana. This waxy substance is known to be rich in bixin and carotenoids, as well as its use as a food coloring agent.

It is known from WO 01/85183 a composition for the prevention and treatment of eye disorders, said composition may include an extract of Bixa orellana as an inhibitor of aldose reductase.

This extract comprises gallic acid and/or pyrogallol, thus it is probably a leaf extract of Bixa orellana, as described by Terashima et al. [Chem. Pharm. Bull. 39 (12), 3346-3347 (1991)], which actually shows its activity as an inhibitor of aldose reductase. In addition, WO 01/85183 shows the inhibition of aldose reductase as part of a mechanism to protect against cataracts and diabetic retinopathy.

WO 01/85183 does not therefore show the effectiveness of an extract from seeds of Bixa orellana for photoprotection of RPE cells.

According to one embodiment of the present invention, the composition of seeds of Bixa orellana is for the treatment of macular degeneration related to age (AMD) in the mammal.

According to another embodiment of the invention, the composition is intended to treat Stargardt's disease and/or retinitis pigmentosa. Stargardt's disease or Stargardt's syndrome is a hereditary disease, involving a bilateral decrease in visual acuity due to atrophy of the macula.

According to another embodiment of the invention, the composition is intended to prevent damage to the retina may be caused by exposure to blue radiation. Blue rays by means of the radiation corresponding to the blue part of the spectrum of visible light, or wavelengths comprised between 435 and 490 nm.

According to one embodiment of the invention, the composition further comprises a derivative of gallic acid and/or a compound of the anthocyanidin family.

The gallic acid derivative can be ellagic acid, either pure or provided as a pomegranate extract. Indeed, pomegranate contains ellagic acid in large quantities (Panichayupakarananta et al., 2010).

The compound of the anthocyanidin family may be cyanidin, either purie or provided in the form of an Acai extract. This plant does indeed contain cyanidin glycosides. Cyanidin may also be provided in the form of an extract of Hibiscus.

The composition of Bixa orellana seeds can be used as a food, a dietary supplement or a medicament.

Dietary supplement means a product containing said compound or extract or enriched in said said compound extracted intended to supplement the diet by providing nutrients beneficial to health as defined by the European Directive 2002/46/EC. For example, a food supplement can be a capsule or tablet to swallow or a powder or small ampulla to be mixed with food and providing beneficial effects on the retina.

A drug means a product containing a precise dose of said compound or said extract as defined by European Directive 65/65/CE ie any substance or composition presented as having properties for treating or preventing the disease in human beings or animal. For example, the drug containing the compound at therapeutic doses may be administered orally in capsule or tablet form or injected intravitreally or administered by any other way to give beneficial effects on the retina.

Another aspect of the invention relates to a composition comprising a derivative of gallic acid and/or a compound of the anthocyanidin family, for photoprotection of the retinal pigmentary epithelium in the mammal.

The gallic acid derivative is preferably ellagic acid, especially purified form or made of an extract of pomegranate. The compound of the anthocyanidin family is preferably cyanidin, either purie or provided in the form of an extract of Acai or Hibiscus. The applications of this alternative composition are the same as those of the previously mentioned composition comprising an extract of Bixa orellana seeds. As well as the latter, the other compositions may be used as a food, a dietary supplement or a medicament.

The invention will be better understood upon reading the following description and examining the accompanying figures. These are for information only and not limiting of the invention.

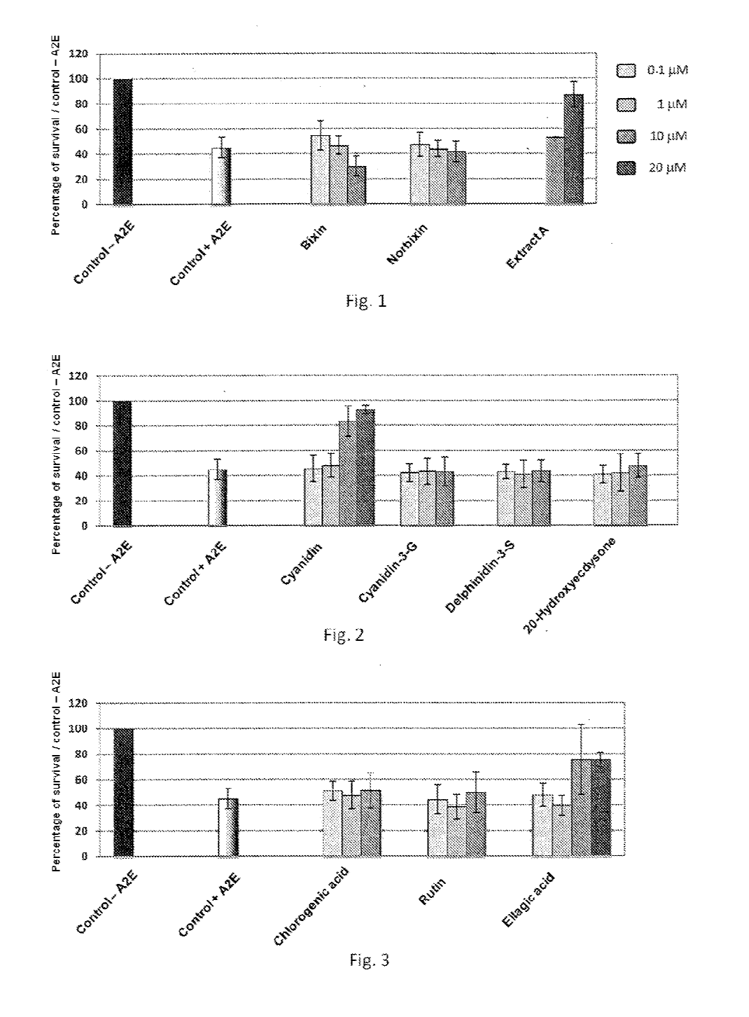

FIG. 1 illustrates the effect of an extract urucum of bixin and norbixin on the protection of the EPR tested for phototoxicity.

FIG. 2 illustrates the effect of chlorogenic acid, rutin and of ellagic acid on the protection of the EPR tested for phototoxicity.

FIG. 3 illustrates the effect of cyanidin, cyanidin-3-glucoside or delphinidin-3-sambubioside and 20-hydroxyecdysone on the protection of the EPR tested for phototoxicity.

### EMBODIMENTS OF THE INVENTION

### I. Preparing an Extract of Bixa Orellana (Extract A)

Extract A is made by stirring the seeds urucum in absolute ethanol (3 L per kg of seeds) for 16 hours. The agitation in alcohol has the effect of detaching the waxy film on the surface lying seeds.

A suspension is obtained. It is sieved to remove urucum seeds. This suspension is then reduced to ⅛th then decanted. A heavy suspension is formed.

The lipid-rich supernatant is eliminated. Maltodextrin is added to the solid deposit and the mixture is spray dried.

The extract contains 16% by weight of bixin. In the following examples, the concentration of the extract is expressed in bixin equivalents.

Extract A is also rich in carotenoids. It also contains other terpenic compounds such as geranylgeraniol and tocotrienols (90% δ and 10% β) and several flavonoids.

The extract has the following features for 100 g (Table 1):

TABLE 1

| | | |
|---|---|---|
| Energetic value | 319.04 | Kcal |
| Carbohydrates (by difference) | 38.10 | g |
| Bixin | 16 | g |
| Protein | 7.7 | g |
| Fat (by hydrolysis) | 1.6 | g |
| Toral fibers | 27.9 | g |
| Sodium | 8.2 | mg |

| | |
|---|---|
| Humidity | 6% |
| Minerals | 4.3% |

According to one embodiment of the invention, the extract A can be subjected to saponification, so as to turn all or part of bixin to norbixin.

II. Activity Assays

The inventors have tested 15 natural substances and extract A on a RPE cellular model of phototoxicity described below (Table 2).

TABLE 2

| Type | Compound name | Source (example) |
|---|---|---|
| Positive controls | Lutein | Spinach |
| | Zeaxanthin | Maize |
| | Resveratrol | Grape |
| Carotenoids | Bixin | Urucum |
| | Norbixin | ″ |
| | Extract A | ″ |
| | Crocetin | Saffron |
| Phenolic acids | Chlorogenic acid | Mate |
| Flavone | Orientin | Açaï |
| Flavonol | Rutin | Buckwheat |
| Flavanone | Naringenin | Lemon |
| Benzopyrane | Ellagic acid | Pomegranate |
| Anthocyanins | Cyanidin 3-glucoside | Açaï |
| | Delphinidin 3-sambubioside | Hibiscus |
| Anthocyanidins | Cyanidin | Açaï* |
| Steroids | 20-Hydroxyecdysone | Quinoa |

*The cyanidin is prepared after acid hydrolysis of its glycosylated forms

To test the photoprotective effect of the test substances, the inventors used a cellular model of induced phototoxicity by treatment with A2E followed by illumination with blue radiation. This model was produced from primary cultures of adult porcine RPE cells. Cell survival was determined by the ratio between the number of living cells and the total number of cells (alive+dead, respectively quantified using specific staining). Image acquisition was performed using a fluorescence microscope controlled by Metamorph software and quantifications were made by processing images acquired by a program dedicated quantification. The experiments were performed in 96-well microplates in quadruplicate and the experiment was repeated at least four times. Cells were treated for 48 hours with these compounds, the last 24 hours in the presence of A2E before induction of phototoxicity. Three concentrations (0.1, 1 and 10 μM) were tested for each compound. Some compounds were further tested at 20 μM to achieve a range of concentrations.

III. Results

The results, presented as averages and standard deviations are expressed as percentage of survival compared to the control without A2E.

The experiments did not show a protective effect of 15 compounds or extract A at concentrations of 0.1 and 1 μM (data not shown for lutein, zeaxanthin, resveratrol, crocetin, naringenin and orientin, and results shown for urucum extract A, cyanidin and ellagic acid in FIGS. 1-3).

In a series of tests (n=5), extract A allowed a significant protection, with cell survival for 20 μM of the order of 93% of the control without A2E, to be compared with that of control +A2E, which is only 45% (FIG. 1).

Among the 15 compounds tested at 10 μM, two provide a cellular protection against phototoxicity.

Notably, 10 μM cyanidin provides a protection in the order of 87% survival compared to the control without A2E. At 20 μM, the same compound provides almost complete protection (FIG. 2).

Ellagic acid also provides protection in the order of 68% compared to the control without A2E. At 20 μM, this compound provides protection similar to that measured at 10 μM, but with a better reproducibility (FIG. 3).

Photoprotective effects expected for lutein, zeaxanthin and resveratrol (“positive controls”) were not observed with these experimental conditions.

### REFERENCES

Bellmann C, Sahel J-A. 2007. Aspects pathogéniques de la dégénérescence maculaire liée à l'âge (DMLA). J. Fr. Ophtalmol. 30 (hors-série 1), 1S11-1S16.
Cohen S Y, Mauget-Faysse M, Oubraham H, Algan, M, Conrath J, Roquet W. 2010. Impact des habitudes nutritionnelles sur la pathologie maculaire évalué par mesure de la densité optique du pigment maculaire. J. Fr. Ophtalmol. 33, 234-240.
Dubernard G, Adam R, Proenca J, Offret H, Labetoulle M. 2006. Alimentation et dégénérescence maculaire liée à l'âge. NPG 6(33), 19-21.
Dunn K C, Aotaki-Keen A E, Putkey F R, Hjelmeland L M. 1996. ARPE-19, a human retinal pigment epithelial cell line with differentiated properties. Exp. Eye Res. 62, 155-169.
Dutot M, Rambaux L, Warnet J M, Rat P. 2008. Modulation du stress oxydant par la myrtille riche en polyphénols sur un modèle de cellules humaines de réfine. J. Fr. Ophthalmol. 31(10), 975-980.
Fajnkuchen F, Cohen S Y. Dégénérescence maculaire liée à l'âge et génétique: données actuelles. J. Fr. Ophtalmol. 31(6), 630-637.
Finnemann S C, Leung L W, Rodriguez-Boulan E. 2002. The lipofuscin component A2E selectively inhibits phagolysosomal degradation of photoreceptor phospholipid by the retinal pigment epithelium. Proc. Nat. Acad. Sci. USA 99: 3842-2847.
Kim S R, Nakanishi K, Itagaki Y, Sparrow J R. 2006. Photooxidation of A2-PE, a photoreceptor outer segment fluorophore, and protection by lutein and zeaxanthin. Exp. Eye Res. 82, 828-839.
Lecerf J-M. 2009. μnutriments: l'exemple de la dégénérescence maculaire liée à l'âge (DMLA). Médecine Maladies Métaboliques 3(5), 496-501.
Lecerf J-M, Desmettre T. 2010. Nutrition et dégénérescence maculaire liée à l'âge. J. Fr. Ophtalmol. 33, 749-757.
Panichayupakarananta P, Issuriya A, Sirikatitham A, Wang W. 2010. Antioxidant assay-guided purification and LC determination of ellagic acid in pomegranate peel. J. Chrom. Sci. 48, 456-459.
Souied E, Le Tien V, Coscas G, Soubrane G. 2007. Vers la prévention de la dégénérescence maculaire liée à l'âge. J. Fr. Ophtalmol. 30(5), 449-455.

1. Composition comprising an extract of seeds of Bixa orellana for photoprotection of the retinal pigmentary epithelium in a mammal.

2. Composition according to claim 1, for its application in the treatment of macular degeneration associated with age (AMD) in a mammal.

3. Composition according to claim 1, for its application in the treatment of Stargardt's disease and/or retinitis pigmentosa.

4. Composition according to claim 1 for preventing retinal damage that could be caused by exposure to blue light of wavelength comprised between 435 and 490 nm.

5. Composition according to claim 1, comprising an acceptable carrier to be ingested or injected into the eye or injected into the blood.

6. Composition according to claim 1 as a medicament or food supplement or food.

7. Composition according to claim 1, further comprising a gallic acid derivative and/or a compound of the family of anthocyanidins.

8. Composition according to claim 7, characterized in that the gallic acid derivative is ellagic acid.

9. The composition according to claim 7, characterized in that the compound of the family of anthocyanidins is cyanidin.

10. A composition comprising a compound of gallic acid and/or a compound of the family of anthocyanidins, for photoprotection of the retinal pigment epithelium in the mammal.

11. Composition according to claim 10, for its application in the treatment of age-related macular degeneration (AMD) in a mammal, or in treating Stargardt's disease and/or retinitis pigmentosa, or to prevent the retinal damage that could be caused by exposure to blue light of wavelength comprised between 435 and 490 nm.

12. Composition according to claim 10, characterized in that the gallic acid derivative is ellagic acid.

13. Composition according to claim 10, characterized in that the compound of the family of anthocyanidins is cyanidin.

14. Composition according to claim 10 as a medicament or food supplement or food.
